(19) United States
(12) Patent Application Publication
(10) Pub. No.: US 2012/0184769 A1
(43) Pub. Date: Jul. 19, 2012

(54) METHOD FOR ISOLATING METHYLGLYCINENITRILE-N,N-DIACETONITRILE

(75) Inventors: Bernd Judat, Ludwigshafen (DE); Alfred Oftring, Bad Duerkheim (DE); Armin Stamm, Nieder-Olm (DE); Friedhelm Teich, Neckarhausen (DE)

(73) Assignee: BASF SE, Ludwigshafen (DE)

(21) Appl. No.: 13/498,825

(22) PCT Filed: Sep. 29, 2010
(86) PCT No.: PCT/IB2010/054382

§ 371 Date: Mar. 28, 2012

(30) Foreign Application Priority Data
Oct. 5, 2009 (EP) 09172246.2

(51) Int. Cl. C07C 253/34

(52) U.S. Cl. 558/441

(57) ABSTRACT

A method for isolating methylglycinenitrile-N,N-diacetonitrile (MGDN) from an aqueous mixture comprising MGDN is provided The method comprises cooling the aqueous mixture in one or more steps In one of these steps the mixture is cooled at a cooling rate of at least 20 K/h from a temperature above the solidification point of MGDN to a temperature below the solidification point of MGDN The method is implemented continuously

The present invention relates to a method for isolating methylglycinenitrile-N,N-diacetonitrile (MGDN) from an aqueous mixture comprising MGDN.

Preferred embodiments are apparent from the dependent claims and from the description.

The aminopolyphosphonates, polycarboxylates or aminopolycarboxylates, such as ethylenediaminotetraacetic acid (EDTA), which are frequently employed as complexing agents, as for example in household cleaning products, are biodegradable only to a low degree. One inexpensive alternative is represented by glycine-N,N-diacetic acid derivatives such as methylglycine-N,N-diacetic acid (MGDA), which are nontoxic and readily biodegradable. The use of MGDA and of related glycine-N,N-diacetic acid derivatives in cleaning products, and also their syntheses, are described in WO-A 94/29421 and U.S. Pat. No. 5,849,950, for example. For cost-effective production of the glycine-N,N-diacetic acid derivatives, exacting requirements are imposed on the yield of the individual synthesis steps and the purity of the isolated intermediates.

MGDA is generally obtained by reacting iminodiacetonitrile (IDN) with acetaldehyde and hydrocyanic acid, or alpha-alaninenitrile with formaldehyde and hydrocyanic acid (referred to as a pH-controlled Strecker reaction), the intermediate MGDN obtained being subjected in a further step to an alkaline hydrolysis with aqueous sodium hydroxide solution. This produces the trisodium salt of MGDA. In order to achieve high MGDA yields, it is desirable to isolate MGDN as an intermediate and to use it as the pure substance in the subsequent hydrolysis step.

MGDN can be prepared in a variety of ways. Generally speaking, MGDN is obtained in the synthesis as an aqueous mixture. It is possible for such mixtures to be composed exclusively of water and MGDN. Aqueous mixtures comprising MGDN, however, may also comprise a series of secondary components. Where, for example, MGDN is prepared by pH-controlled Strecker reaction, iminodiacetonitrile (IDN) can either be used as the crystalline raw material or else generated by a pH-controlled reaction of urotropine with HCN in aqueous solution and reacted, without itself being isolated, to give the MGDN. In that case, secondary components present in the aqueous MGDN mixture include ammonium sulfate, acetaldehyde cyanohydrin, formaldehyde cyanohydrin, methylenebisiminodiacetonitrile (MBIDN), nitrilotriacetonitrile (NTN), dimethylglycineacetonitrile (DMGN), acetaldehyde, hydrocyanic acid, and unconverted reactants.

The solubility of MGDN in water is highly temperature-dependent. Thus, at 10° C., only about 0.5% by weight of MGDN can still be dissolved in water or an aqueous ammonium sulfate solution. At around 60° C., the solubility is still about 5% by weight. Above about 60° C., an emulsion is formed of MGDN and its aqueous solution with more than 5% by weight of dissolved MGDN. A complete phase diagram of the water-MGDN system is unknown.

MGDN has a solidification point, which, however, is not constant but is instead dependent, for example, on all of the components present in the mixture, such as, for example, on the salt content of the mixture in which MGDN is present, on the concentration of MGDN in the solvent, and on the nature and amount of the secondary products present in the mixture. The solidification point of pure MGDN is around 82° C.; the solidification point of MGDN in aqueous mixtures is generally between 55 and 65° C.

In view of the high temperature dependency of the solubility of MGDN in water, MGDN can be isolated from aqueous mixtures by cooling crystallization and subsequent solid/liquid separation. When an aqueous solution or emulsion of MGDN is cooled, MGDN in solid form is obtained as fine, acicular crystals, which may agglomerate. One of the problems affecting the crystallization of MGDN is the inclusion of impurities.

In U.S. Pat. No. 5,849,950, example 2, MGDN is crystallized out of the crude product mixture from the reaction of HCN, formaldehyde, and alaninenitrile—which is generated in a preliminary step in situ from acetaldehyde, HCN and ammonia—by cooling of the product mixture.

EP 1883623 describes a method for isolating MGDN that involves cooling an aqueous emulsion of MGDN in at least two steps. The cooling step from above to below the solidification point of MGDN in that case takes place very slowly, with an average cooling rate of less than 5 K/h, in order to achieve slow crystallization of the MGDN.

It was an object of this invention to provide an improved method for removing MGDN from the aqueous crude product mixtures obtained during its preparation, said method enabling high yields and purities of MGDN. A further intention was to provide a method that affords an extremely consistent quality of the product. A further objective was to achieve an extremely high space/time yield.

The object has been achieved by the method identified above, comprising cooling the aqueous, MGDN-comprising mixture in one or more steps, in one of these steps the mixture being cooled at a cooling rate of at least 20 K/h from a temperature above the solidification point of MGDN to a temperature below the solidification point of MGDN, the method being implemented continuously.

The isolation of constituents from a liquid mixture through formation of precipitates is frequently referred to by the skilled worker as “crystallization”. The terms “crystals”, “crystallization” and “crystalline” in the sense of this invention therefore refer not always only to compounds whose molecules are regularly disposed in a crystal lattice, but also comprise, generally, all solids and precipitates which are deposited from the aqueous mixtures described, even if they have amorphous regions. Depending on the boundary conditions, for example, the terms may also be used to apply to the solidification of a drop of liquid.

A continuous method is understood in the sense of this invention to refer to a crystallization in which a mass flow of MGDN-rich crude mixture is fed over a relatively long time period, as for example over several hours, days, weeks or years, into the apparatus in which the method of the invention is carried out, without it being necessary to interrupt the crystallization or to empty the crystallizer or crystallizers. At the same time, with a continuous method, a mass flow of cooled final mixture that is of equal size or substantially equal size on average is removed from the crystallizer over the same time period. The mass flows may be constant over the time period, or vary, in terms of their amount per unit time. By “vary” is also meant the interruption of the mass flows for a short time, by means, for example, of cyclical opening and closing of valves. According to one of the preferred embodiments, the mass flows are substantially constant.

Crystallizers are the apparatus in which the cooling steps, described below, of the aqueous, MGDN-comprising mixture are carried out.

In accordance with the invention, the isolation of MGDN from the aqueous mixture is carried out in one or in two or more cooling steps. A cooling step is the operation in which the temperature of the aqueous mixture is lowered from an initial temperature to a final temperature. The difference between the two temperatures may amount, for example, to at least one kelvin, and is usually at least two kelvins. In one preferred variant, the temperature difference is at least five or at least ten kelvins. Generally speaking, the method of the invention comprises one to five cooling steps, preferably two to three.

In the course of a cooling step, the aqueous mixture changes in its composition. Prior to a cooling step, the aqueous mixture is warmer and there is a greater amount of MGDN present in solution or emulsion in the aqueous mixture. The aqueous mixture in its condition prior to a cooling step is also referred to in this description as the “crude mixture”. In contrast, the aqueous mixture after a cooling step is also referred to as the “final mixture”. This latter mixture is colder and now comprises only a smaller amount of MGDN in dissolved form.

If the solidification point of MGDN for a particular aqueous mixture is unknown, it may be determined, for example, by using microscopic observation to ascertain the temperature at which a drop of MGDN emulsified in water becomes solid. One of the cooling steps, in accordance with the invention, constitutes the phase transition of MGDN, emulsified in water, from liquid to solid at the solidification point. This cooling step is particularly important for the method, since it is during this cooling step that the major fraction of the MGDN undergoes crystallization. The initial temperature for this cooling step is generally at least 0.5 K above the solidification point, preferably at least one kelvin, more preferably at least two kelvins, very preferably at least five kelvins, with particular preference at least 10 kelvins. In general, the temperature of the crude mixture may be at any desired level above the solidification point. Generally speaking, however, it is not above the boiling point of the crude mixture at atmospheric pressure, since otherwise the method of the invention would have to be performed under increased pressure. It is preferably not higher than 90° C. The final temperature of the cooling step is generally at least 0.5 K below the solidification point of MGDN, preferably at least 1 K, more preferably at least 2 K, very preferably at least 5 K. It is likewise possible to choose a final temperature of more than 10 K below the solidification point.

The cooling step via the solidification point may be preceded and followed by an arbitrary number of cooling steps. Preceding cooling steps generally have the purpose of approximating the temperature of the crude mixture to the solidification point. Subsequent cooling steps serve primarily to increase the yield by lowering the solubility of MGDN in water.

For the subsequent cooling steps, the final temperature of the preceding step is preferably the initial temperature of the following step. The temperature difference in the subsequent cooling steps is generally more than 10 K, preferably more than 20 K and more preferably more than 30 K. In one particularly preferred embodiment, the temperature difference in these cooling steps is 40 to 50 K.

In less-preferred embodiments of the method of the invention, however, the aqueous mixture, after a cooling step, may also be subjected to other steps and may, for example, be heated again. The performance of seeding loops is less preferred for the method of the invention.

In one preferred embodiment, the crystallization is carried out in two cooling steps, the crude mixture being cooled in the first step from a temperature above the solidification point of MGDN to a temperature below this temperature. In a second step, the temperature is then significantly reduced once again, in order to raise the yield. Generally speaking, the final temperature of the last cooling step is from −10° C. to 50° C., preferably from 0° C. to 30° C., and more preferably from 5° C. to 15° C.

The cooling rate in the sense of this invention means in each case the average temperature gradient over the time from the initial temperature to the final temperature of a cooling step. In accordance with the invention, the cooling rate during the cooling step from above to below the solidification point of MGDN is at least 20 K/h, preferably at least 50 K/h, more preferably at least 100 K/h. It is possible to carry out all the cooling steps at the same cooling rate, but they may also differ in each cooling step. In one preferred embodiment of the invention, the crude mixture is cooled by adding the crude mixture to aqueous mixture which has the final temperature or a temperature slightly below it. The volume of the aqueous, MGDN-comprising mixture introduced initially is preferably large by comparison with the mass flow of crude mixture. An embodiment of this kind may result in cooling rates of more than 1000 K/h, and cooling rates of more than 3000 K/h, 5000 K/h or 7500 K/h may be realized in this way.

The manner in which the cooling of the crystallizers is carried out is not critical to the invention. Possible techniques of cooling include, for example, cooling by heat exchangers or evaporation of water from the aqueous mixture. Cooling of the crystallizers by heat exchangers may offer the advantage that little foam is formed in the crystallizers. Evaporation of water from the aqueous mixture may offer the advantage of reduced formation of crystal seeds and of deposits on the walls of the crystallizer. The evaporation of water may take place with the amount of water constant, the water evaporated being returned under reflux. Alternatively, water may also be removed from the aqueous mixture in such a way that the amount of water is reduced, thus resulting in a concentration of the MGDN in the aqueous mixture. The techniques may also be combined with another.

Cooling may also take place by the cooling of the aqueous mixture in a tubular crystallizer with different temperature zones along the flow section. Tubular crystallizers may be operated with or without scratching members that keep the walls of the crystallizer free from deposits. Another possibility is the use of cooling disks. Cooling techniques of this kind are known per se to the skilled worker.

It is preferred, during the cooling step from above to below the solidification point of MGDN, to introduce mechanical energy. In one preferred embodiment, mechanical energy is introduced during each cooling step. It is also possible to introduce mechanical energy throughout the method for isolating MGDN. Generally speaking, an energy is introduced at least with an average specific energy input of 0.5 kW/m³, preferably at least 0.8 kW/m³, more preferably at least 1 kW/m³, and with particular preference 1.5 kW/m³. There is in principle no upper limit on the input of energy, but it will be guided by the specifications of the apparatus and by practical considerations such as, for example, the size and construction of the crystallizer, or, possibly, the nature of the stirrer, economic considerations, and the target crystal size. The introduction of mechanical energy by means of the method of the invention means that only a little aqueous mixture, which may comprise all of the secondary components of MGDN preparation, is included as an impurity in crystal agglomerates. In contrast to crystallizates comprising crystal agglomerates from other methods, MGDN isolated by the method of the invention has a reduced brownish coloration. It is a surprising result of the method of the invention that, in spite of the high cooling rates, the formation of crystal agglomerates can be effectively restricted, since the liquid MGDN droplets emulsified in the mixture have a high viscosity and stickiness. As a result of the method of the invention, moreover, there are reductions in the tendency of the emulsified MGDN droplets to deposit on surfaces, and in the tendency to form encrustations on the walls of the crystallizer.

By appropriately setting the mechanical energy input it is also possible to control the size of the solid MGDN particles that form, in such a way that the particles obtained are large enough to be effectively filtered but small enough so that for subsequent reactions they dissolve again effectively in the reaction medium concerned. In general, particles having average sizes in the range from 100 to 1000 μm have good handling qualities. Particles which have smaller or larger average sizes may likewise be prepared. They may necessitate particular apparatus for handling. Techniques for determining the particle sizes are known to the skilled worker.

Moreover, the energy input influences the extent to which deposits are formed on the walls. A precise correlation, for example, of defined particle sizes with defined mechanical energies is not possible, since it is greatly dependent on the specifications of the apparatus and on the other boundary conditions, especially the composition of the crude mixture. Generally speaking, the introduction of a high mechanical energy tends to produce smaller particles and less deposition. The way in which the energy is introduced into the system is not critical. One possible energy source, for example, is ultrasound, or spraying of the aqueous mixture through nozzles. The input of energy is preferably accomplished by mechanical stirring. Where the major part of the mechanical energy is introduced by stirring, particular preference for this purpose is given to high-speed stirrer types such as propeller stirrers, inclined-blade stirrers, disk stirrers or toothed-disk stirrers. The stirring operation in the crystallizer preferably generates a turbulent flow. Depending on crystallizer construction, it may be useful to use flow disruptors.

The individual cooling steps can be carried out in different types of crystallizers. Their nature is not critical to the method. Possible types of crystallizer are, for example, forced-circulation, guide-tube or stirred-tank crystallizers. The latter may have internals, though it is preferred to use them free from internals.

In one preferred embodiment, the method of the invention is performed such that each cooling step is carried out in a stirred tank as crystallizer, into which a continuous flow of crude MGDN mixture is added and a continuous flow of cooled, MGDN-depleted mixture is taken off. With very particular preference, the method of the invention is carried out in a cascade of stirred tanks. With more particular preference, the cascade of stirred tanks comprises two stirred tanks.

The addition of the crude mixture may be made in principle at any location in the crystallizer. Preferably, however, it is accomplished in such a way that the crude mixture added immediately experiences high shearing forces. Possibilities, for example, accordingly include the addition through a valve directly into the aqueous mixture introduced initially, or by means of submersed addition, as for example via a stirrer—where present. Less preferred is dropwise addition from above onto the surface of the aqueous mixture introduced initially. The discharging of the final mixture may occur in principle at any location in the crystallizer. Preferably, the cooled final mixture is withdrawn from the underside of the crystallizer. The average residence time of the aqueous mixture in the crystallizer is in general at least 10 minutes, preferably 20 minutes, very preferably 30 minutes, and with particular preference at least 60 minutes. In one preferred variant, the method comprises two cooling steps, each carried out in a stirred tank.

The removal of the solid MGDN from the aqueous mixture may take place by methods which are known to the skilled worker, as for example by means of a hydrocylinder for solid/liquid separation, or a belt filter. After each cooling step it is possible to remove crystallized MGDN from the aqueous mixture. It is likewise possible to remove crystallized MGDN only after the last cooling step. Removal may take place continuously or batchwise.

The method of the invention is suitable for isolating MGDN from an aqueous mixture, irrespective of the way in which said mixture has been produced. It is possible to provide the aqueous mixture by mixing solid MGDN with water. The aqueous mixture is preferably obtained by preparing MGDN in situ in an aqueous medium, adjusting, if desired, the proportion of MGDN to solvent, and using this aqueous mixture to perform the method of the invention for isolating MGDN.

The aqueous, MGDN-comprising mixture from which MGDN is isolated with the method of the invention may be obtained, for example, by
1. reacting iminodiacetonitrile (IDN) with HCN and acetaldehyde in aqueous solution—iminodiacetonitrile can be obtained in a preceding stage from urotropine and hydrocyanic acid or from formaldehyde cyanohydrin and ammonia in the form of an aqueous emulsion; or
2. reacting alaninenitrile with HCN and formaldehyde in aqueous solution—alaninenitrile can be obtained in a preceding stage from acetaldehyde, HCN, and ammonia, or acetaldehyde cyanohydrin and ammonia.

The conditions under which an aqueous mixture comprising MGDN can be obtained are known to the skilled worker from EP 1883623, for example.

Prior to cooling below the solidification point of MGDN, the aqueous, MGDN-comprising mixture comprises preferably 5% to 50% by weight of MGDN, more preferably 10% to 40% by weight, and very preferably 15% to 30% by weight of MGDN. MGDN is present partly in solution. Where the MGDN content of the aqueous mixture exceeds its solubility, MGDN is present partly as an emulsion.

The solvent of the aqueous mixture is composed substantially of water. Generally speaking, at least 70% by weight of the solvent used is water, preferably at least 85% by weight, more preferably 95% by weight. In one especially preferred embodiment, the aqueous mixture comprises no further solvents apart from water.

In less-preferred embodiments of the invention, the aqueous mixture comprises organic solvents as well as water. These solvents must be miscible with water under the conditions of the method. Examples of suitable organic solvents include alcohols such as methanol or ethanol, or other polar solvents.

The method of the invention makes it possible in general to isolate MGDN with a yield of at least 70%. It is preferred to isolate at least 85%, more preferably 90%, and very preferably 95% of the MGDN used, based on the amount of MGDN in the aqueous mixture prior to the first cooling step.

Following isolation, solid or redissolved MGDN may be subjected to further purification steps. It is possible to lower the color number of the MGDN by adsorption on activated carbon or by means of bleaching operations. Possible bleaching operations include, for example, photobleaching, photooxidation or ozonolysis. Bleaching operations of this kind are known per se to the skilled worker.

The purity of the isolated MGDN is generally more than 98% by weight.

A particular advantage of the method of the invention is a very high space/time yield.

### EXAMPLES

The examples which follow are intended to illustrate the properties of this invention, but without restricting it.

“Parts”, percentages or ppm in this specification are understood as weight fractions unless indicated otherwise.

### Example 1

### Preparation of an Aqueous Crude MGDN Mixture

IDN was prepared by reacting a solution of 173.9 g (1.241 mol) of urotropine in 535 g of water with 210.9 g (7.814 mol) of hydrocyanic acid. The pH was regulated at 5.8-5.9 by metered addition of a total of 188 g of 50% strength by weight sulfuric acid.

The result was 1090 g of an aqueous solution containing 336.3 g (3.54 mol) of IDN (95% yield, based on formaldehyde). The pH of the solution was adjusted to 1.8 by addition of 60 g (0.306 mol) of 50% strength by weight sulfuric acid, and its temperature to 60° C. Then, over the course of 75 minutes, 105.1 g (3.894 mol) of HCN and 171.6 g (3.894 mol) of acetaldehyde were metered in. The temperature climbed to around 80° C. Stirring was continued at 80° C. for 60 minutes more. The pH dropped during the addition of reactants and during the subsequent reaction to around 1.2. The product was around 1440 g of an approximately 36% strength by weight MGDN emulsion which contained 515 g of MGDN (3.469 mol; 98% yield, based on IDN). The crude MGDN mixture obtained had a dark brown color.

### Examples 2 to 3

### Continuous Two-Stage Crystallization of MGDN

For the following examples, crude MGDN mixture was used that had been prepared in accordance with example 1.

For the experiments, an experimental plant was constructed that consisted of two stirred tanks. From a stirred reservoir conditioned to a temperature of 70° C., the crude MGDN mixture was pumped continuously into the first stirred tank. Transfer to the second stirred tank took place under level control, by cyclical opening and closing of the bottom drain valve of the first stirred tank. In order to prevent encrustations in the drain pipe, each opening of the bottom drain valve and running of the suspension portion into the second stirred tank was followed by a flushing cycle with hot water. The flush solution was removed from the system via a three-way tap. As a result of this measure, the two-stage plant could be operated continuously.

### Example 2

### Continuous Two-Stage Crystallization of MGDN

Aqueous MGDN mixture with an MGDN content of 21.0% by weight was introduced and heated to a temperature of 55° C. The mixture was stirred with a propeller stirrer at 720 revolutions per minute. Metered into this mixture continuously over a time period of around six hours was aqueous crude MGDN mixture with a temperature of 70° C. After around five hours, a steady state was established. The average residence time was around an hour. Large crystals of MGDN were formed in pulses.

As described above, the aqueous MGDN mixture was removed from the stirred tank and passed into a second, identical stirred tank, which likewise contained aqueous MGDN mixture and was thermostatted at 13° C. From this vessel, again under level control, aqueous MGDN mixture was withdrawn.

Crystallized MGDN was removed from the aqueous mixture by pressure filtration and was washed with water.

The yield of crystalline MGDN was more than 95%, based on the MGDN present in the aqueous mixture introduced initially.

The crystalline MGDN obtained in this way was analyzed by HPLC. The amount of IDN was less than 0.01%, and lay below the detection limit. The MGDN obtained had a pale beige color.

### Example 3

### Continuous Two-Stage Crystallization of MGDN

Aqueous MGDN mixture with an MGDN content of 19.9% by weight was introduced and heated to a temperature of 36° C. The mixture was stirred with a propeller stirrer at 720 revolutions per minute. Metered into this mixture continuously over a time period of around six hours was aqueous crude MGDN mixture with a temperature of 70° C. The average residence time was around an hour. Large crystals of MGDN were formed in pulses.

As described above, the aqueous MGDN mixture was removed from the stirred tank and passed into a second, identical stirred tank, which likewise contained aqueous

MGDN mixture and was thermostatted at 12° C. From this vessel, again under level control, aqueous MGDN mixture was withdrawn.

Crystallized MGDN was removed from the aqueous mixture by pressure filtration and was washed with water.

The yield of crystalline MGDN was more than 95%, based on the MGDN present in the crude mixture.

The crystalline MGDN obtained in this way was analyzed by HPLC. The amount of byproduct IDN was less than 0.01%, and lay below the detection limit. The MGDN obtained had a pale beige color.

1. A method for isolating methylglycinenitrile-N,N-diacetonitrile (MGDN) from an aqueous mixture comprising MGDN, the method comprising cooling the mixture in one or more steps, to obtain isolated MQDN, wherein one step comprises cooling the mixture at a cooling rate of at least 20 K/h from a temperature above a solidification point of MGDN to a temperature below the solidification point of MGDN, and wherein the method is continuous.

2. The method of claim 1, wherein each cooling step comprises cooling the mixture at a cooling rate of at least 20 K/h.

3. The method of claim 1, further comprising introducing mechanical energy having an average specific energy input of at least 0.5 kW/m³ into the aqueous mixture during the cooling step from a temperature above the solidification point of MGDN to a temperature below the solidification point of MGDN.

4. The method of claim 1, wherein at least one cooling step is performed such that the mixture is added to a second, colder, aqueous mixture comprising MGDN, having a temperature below the solidification point of MGDN.

5. The method of claim 1, wherein a temperature of the mixture after a final cooling step is −10° C. to 50° C.

6. The method of claim 1, comprising cooling the mixture in two steps.

7. The method of claim 1, wherein the isolated MGDN has a purity of at least 90%.

8. The method of claim 1, wherein each cooling step comprises cooling the mixture by at least 5 K.

9. The method of claim 1, wherein the temperature above the solidification point of MGDN is at least 0.5 K above the solidification point.

10. The method of claim 1, wherein the temperature above the solidification point of MGDN is at least 10 K above the solidification point.

11. The method of claim 1, wherein the temperature above the solidification point of MGDN is not higher than 90° C.

12. The method of claim 1, wherein the temperature below the solidification point of MGDN is at least 0.5 K below the solidification point.

13. The method of claim 1, wherein the temperature below the solidification point of MGDN is at least 5 K below the solidification point.

14. The method of claim 1, wherein a temperature of the mixture after a final cooling step is 0° C. to 30° C.

15. The method of claim 1, wherein a temperature of the mixture after a final cooling step is 5° C. to 15° C.

16. The method of claim 1, wherein one step comprises cooling the mixture at a cooling rate of at least 50 K/h from a temperature above the solidification point of MGDN to a temperature below the solidification point of MGDN.

17. The method of claim 1, wherein one step comprises cooling the mixture at a cooling rate of at least 100 K/h from a temperature above the solidification point of MGDN to a temperature below the solidification point of MGDN.

18. The method of claim 3, wherein the mechanical energy has an average specific energy input of at least 1.5 kW/m³.

19. The method of claim 1, wherein prior to the cooling below the solidification point, the aqueous mixture comprises 5% to 50% by weight of MGDN.

20. The method of claim 1, wherein the isolated MGDN has a purity of at least 98%.
